(19) United States
(12) Patent Application Publication
(10) Pub. No.: US 2007/0099291 A1
(43) Pub. Date: May 3, 2007

(54) Ambient pressure monitor and method of preparing biological specimens

(75) Inventor: Douglas A. Tenney, North Reading, MA (US)

Correspondence Address: VISTA IP LAW GROUP LLP, 12930 Saratoga Avenue, Suite D-2, Saratoga, CA, 95070, US

(73) Assignee: Cytyc Corporation, Marlborough, MA (US)

(21) Appl. No.: 11/267,529

(22) Filed: Nov. 3, 2005

(51) Int. Cl. C12M 1/34

(52) U.S. Cl. 435/287.9; 422/65

(57) ABSTRACT

A filtration based biological specimen collection and transfer device includes a vacuum source, a vacuum conduit configured to connect the vacuum source to a filter cartridge, an internal pressure monitor configured to measure an apparent pressure inside of the filter cartridge, an internal pressure monitor conduit configured to connect the internal pressure monitor to the filter cartridge, an ambient pressure monitor configured to measure an ambient pressure, and a controller operatively connected to the vacuum source, the internal pressure monitor, and the ambient pressure monitor. The controller is configured to calculate a true pressure inside of the filter cartridge based on a measured apparent pressure and a measured change in the ambient pressure. A method of collecting a biological specimen comprising measuring an apparent pressure inside of a filter cartridge, measuring an ambient pressure, calculating a true pressure inside of the filter cartridge using the respective measured apparent pressure and ambient pressure, deactivating the vacuum source when the true pressure inside of the filter cartridge falls below a predetermined pressure, allowing the true pressure inside of the filter to return to approximately the ambient pressure, and calculating a membrane occlusion percentage.

### FIELD OF INVENTION

The present invention generally relates to devices and methods for preparing biological specimens, and more particularly, to filtration based devices and methods for preparing biological specimens.

### DESCRIPTION OF RELATED ART

Many medical diagnostic tests, such as pap smears, require a physician to collect cells by brushing and/or scraping a skin or mucous membrane in a target area with an instrument. The collected cells are typically smeared (“fixed”) onto a slide, and stained to facilitate examination under a microscope by a cytotechnologist and/or pathologist. For example, a pathologist may employ a polychrome technique, characterized by staining the nuclear part of the cells, to determine the presence of dysplasia or neoplasia. The pathologist may also apply a counter-stain for viewing the cytoplasm of the cells. Because the sample may contain debris, blood, mucus and other obscuring artifacts, the test may be difficult to evaluate, and may not provide an accurate diagnostic assessment of the collected sample.

Cytology based on the collection of the exfoliated cells into a liquid preservative offers many advantages over the traditional method of smearing the cells directly onto the slide. A slide can be prepared from the cell suspension using a filter transfer technique, as disclosed in U.S. Pat. Nos. 6,572,824, 6,318,190, 5,772,818, 5,364,597 and 5,143,627, which are expressly incorporated herein by reference.

Filter transfer methods generally start with a collection of cells suspended in a liquid. These cells may be collected and dispersed into a liquid preservative or they may naturally exist in a collected biological liquid. Dispersion in liquid preservatives containing methanol, such as PreservCyt™ solution, breaks up mucus and lyses red blood cells and inflammatory cells, without affecting the cells of interest. The liquid is passed through a filter with a fixed diameter aperture covered by a membrane to concentrate and collect the cells. Debris, such as lysed blood cells and dispersed mucus, which flow through the pores of the membrane, are not collected on the membrane and are greatly reduced in the collected specimen by the combined methods of dispersion and filtering. Then the cells collected on the membrane are transferred onto a slide for further processing, such as visual examination.

Filter transfer methods collect cells on the surface of a membrane mounted on a filter cartridge through a series of “sips”. Each “sip” starts with a vacuum source applying a negative pressure to the inside of a filter cartridge. When the vacuum source is activated, the pressure inside the filter cartridge is temporarily lowered. When a desired pressure change is detected, the vacuum source is deactivated. The pressure drop then “decays” as the interior pressure equilibrates with the ambient pressure as cell containing liquid is drawn across the membrane and into the filter cartridge. The “decay” of this temporary pressure drop, specifically the change in pressure inside of the filter cartridge over time, is then used to calculate the amount of cells collected on the membrane of the filter, or the “membrane occlusion percentage”. The plot of pressure over time during a sip is called a “sip curve” and the change in change in pressure over time is the slope of the “sip curve”.

The pressures used to plot the “sip curve” involve measurements with precisions of ±0.001 P.S.I. The pressure measurements are performed with differential pressure sensors so that they can be referenced to atmosphere. Unfortunately, inaccuracies can arise with sudden ambient pressure changes, such as result from doors being opened and closed or an air conditioning systems going on or off, once sipping has commenced. These changes in ambient pressure result in a smooth exponentially decaying curve becoming jagged and make determination of the time to reach equilibrium and the slope of the curve difficult. Software filters are available to reduce the sensitivity of the system to fluctuations in ambient pressure but software filters are limited due to the fact that the frequency band of the fluctuations overlaps the frequency band required for monitoring a “sip”.

### SUMMARY OF THE INVENTION

In one embodiment, a filtration based biological specimen collection and transfer device comprises a vacuum source, a vacuum conduit configured to connect the vacuum source to a filter cartridge, an internal pressure monitor configured to measure an apparent pressure inside of the filter cartridge, an internal pressure monitor conduit configured to connect the internal pressure monitor to the filter cartridge, an ambient pressure monitor configured to measure an ambient pressure, and a controller operatively connected to the vacuum source, the internal pressure monitor, and the ambient pressure monitor. The controller is configured to calculate a true pressure inside of the filter cartridge based on a measured apparent pressure and a measured change in the ambient pressure. The filter cartridge comprises a tubular body with a proximal end and a distal end, and a membrane disposed on the distal end of the tubular body, wherein the proximal end is configured to form airtight seals with the vacuum conduit and the internal pressure monitor conduit. The controller is configured to calculate a true pressure inside of the filter cartridge based on a measured apparent pressure and a measured change in the ambient pressure, and to calculate a membrane occlusion percentage based on the calculated true pressure.

In some embodiments, the controller comprises an associated memory, wherein the memory is configured to store true pressures calculated based on respective measured apparent pressures and respective measured changes in the ambient pressure, and the controller calculates the membrane occlusion percentage based on a change over time of the calculated true pressures. In various embodiments, the ambient pressure monitor comprises a first opening connecting the ambient pressure monitor to an ambient atmosphere, a second opening connecting the ambient pressure monitor to the ambient atmosphere, and a selectively closable door configured to close the second opening, wherein the ambient pressure monitor is configured to measure the ambient pressure at an instant when the door shuts. The controller is operatively connected to the door and configured to shut the door when the vacuum source is activated. The controller comprises a memory configured to record a baseline ambient pressure measured by the ambient pressure monitor, wherein the controller is configured to calculate an ambient pressure change using the baseline ambient pressure and a respective measured ambient pressure.

In one embodiment, a filtration based biological specimen collection and transfer system comprises a biological sample container handler configured for use with a biological sample container, a filter cartridge handler configured for use with a filter cartridge, a biological specimen slide handler configured for use with a biological specimen slide, a vacuum source, a vacuum conduit configured to connect the vacuum source to a filter cartridge, an internal pressure monitor configured to measure an apparent pressure inside of the filter cartridge, an internal pressure monitor conduit configured to connect the internal pressure monitor to the filter cartridge, an ambient pressure monitor configured to measure an ambient pressure, and a controller operatively connected to the biological sample container handler, the filter cartridge handler, the biological specimen slide handler, the vacuum source, the internal pressure monitor, and the ambient pressure monitor. The controller is configured to calculate a true pressure inside of the filter cartridge based on a measured apparent pressure and a measured change in the ambient pressure. The filter cartridge comprises a tubular body with a proximal end and a distal end, and a membrane disposed on the distal end of the tubular body, wherein the proximal end is configured to form airtight seals with the vacuum conduit and the internal pressure monitor conduit. The controller is configured to calculate a true pressure inside of the filter cartridge based on a measured apparent pressure and a measured change in the ambient pressure, and to calculate a membrane occlusion percentage based on the calculated true pressure.

In one embodiment, a method of collecting a biological specimen comprises providing a liquid biological sample in a biological sample container, providing a filter cartridge forming a chamber and having a distal end with a membrane disposed thereon, positioning the distal end of the filter cartridge and the membrane inside of the liquid biological sample in the biological sample container, activating a vacuum source to lower the pressure of the chamber, measuring an apparent pressure of the chamber, measuring an ambient pressure, calculating a true pressure of the chamber using the respective measured apparent pressure and ambient pressure, deactivating the vacuum source when the true pressure of the chamber falls below a predetermined pressure, allowing the true pressure inside of the filter to return to approximately the ambient pressure, calculating a membrane occlusion percentage, and repeating the above steps until the calculated membrane occlusion percentage is greater than a predetermined number.

In some embodiments, the method of collecting a biological specimen comprises measuring a baseline ambient pressure, and calculating an ambient pressure change using the baseline ambient pressure and the respective ambient pressure. In various embodiments, the method also comprises storing true pressures calculated over time and calculating the membrane occlusion percentage using a change in true pressure over time. In some embodiments, the method also comprises collecting an approximately one cell thick layer of cells on the membrane and transferring the layer of cells to a biological specimen slide.

### BRIEF DESCRIPTION OF THE DRAWINGS

In order to better understand and appreciate the invention, reference should be made to the drawings and accompany detailed description, which illustrate and describe exemplary embodiments thereof. For ease in illustration and understanding, similar elements in the different illustrated embodiments are referred to by common reference numerals. In particular:

FIG. 1 is a schematic view of an exemplary filtration based biological specimen collection and transfer device according to one embodiment of the invention;

FIG. 2A is a schematic view of an ambient pressure monitor according to one embodiment of the invention;

FIG. 2B is another schematic view of the ambient pressure monitor of FIG. 2A;

FIG. 3 is a schematic view of an exemplary filtration based biological specimen collection and transfer system according to one embodiment of the invention;

FIG. 4 is another schematic view of the exemplary filtration based biological specimen collection and transfer system of FIG. 3;

FIG. 5 is an exemplary practical sip curve for a filtration based biological specimen collection and transfer system; and

FIG. 6 is an exemplary theoretical sip curve for a filtration based biological specimen collection and transfer system.

### DETAILED DESCRIPTION OF THE ILLUSTRATED EMBODIMENTS

In the following description of the illustrated embodiments, it will be understood by those skilled in the art that the drawings and specific components thereof are not necessarily to scale, and that various structural changes may be made without departing from the scope or nature of the various embodiments.

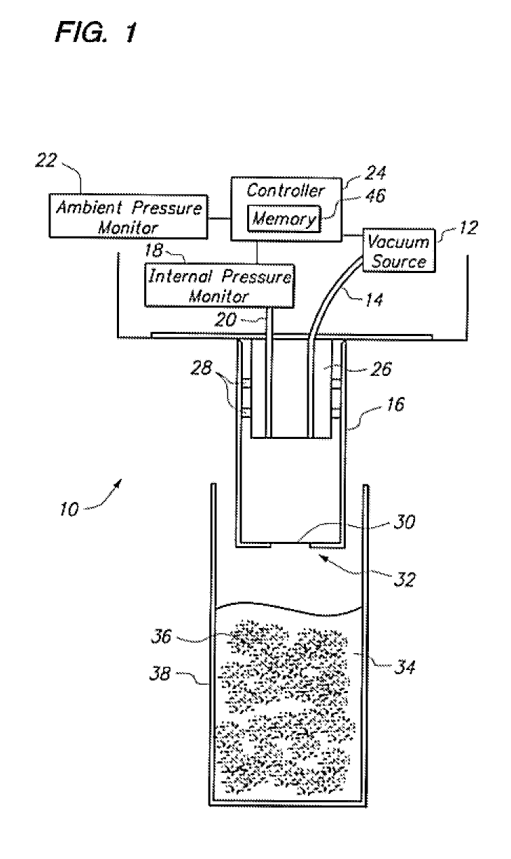

Referring to FIG. 1, a biological specimen collection and transfer device 10 is shown. In this embodiment, the biological specimen collection and transfer device 10 includes a vacuum source 12, a vacuum conduit 14, a filter cartridge 16, an internal pressure monitor 18, an internal pressure monitor conduit 20, an ambient pressure monitor 22, and a controller 24.

The vacuum conduit 14 and the internal pressure monitor conduit 20 are connected to the vacuum source 12 and the internal pressure monitor 18, respectively. The distal ends of the vacuum conduit 16 and the internal pressure monitor conduit 20 traverse a stem 26, which is connected, with air tight seals 28, to the filter cartridge 16. The internal pressure monitor 18 and the ambient pressure monitor 22 both have a sensitivity of ±0.001 P.S.I.

The filter cartridge 16 has a membrane 30 at an open distal end 32, which is configured to be submerged into a liquid 34 containing a biological sample 36, such as collected cervical cells. A biological sample container 38 holds the liquid 34.

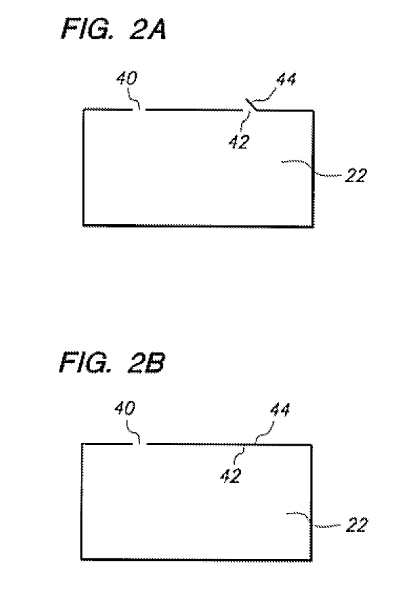

Referring to FIG. 2A, the ambient pressure monitor 22 includes a first opening 40 and a second opening 42, which are both open to atmosphere. The ambient pressure monitor 22 also comprises a door 44, configured to close the second opening 42. When the biological specimen collection and transfer device 10 is not sipping, both openings 40/42 are open to atmosphere. At the beginning of a sip, the door 44 closes the second opening 42, configuring the ambient pressure monitor 22 to detect an ambient pressure.

The controller 24 is connected to the vacuum source 12, the internal pressure monitor 20, and the ambient pressure monitor 22. The controller 24 is configured to communicate with the vacuum source 12, the internal pressure monitor 20, and the ambient pressure monitor 22. The controller 24 includes a memory 46, which is configured to store information received from the internal pressure monitor 20 and the ambient pressure monitor 22.

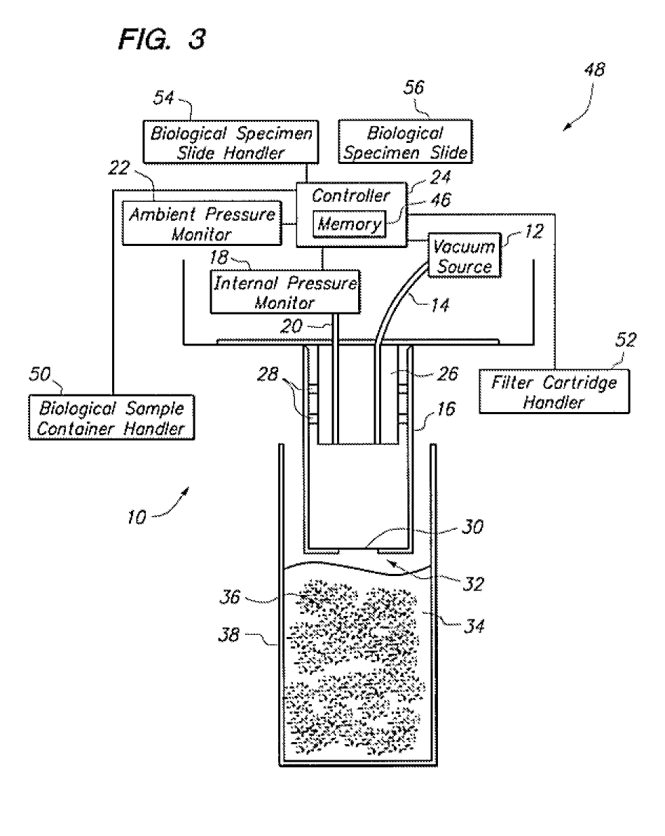

Referring to FIG. 3, a biological specimen collection and transfer system 48 is shown. In this embodiment, the biological specimen collection and transfer system 48 includes all elements of a biological specimen collection and transfer device 10 as described above, as well as a biological sample container handler 50, a filter cartridge handler 52, and a biological specimen slide handler 54.

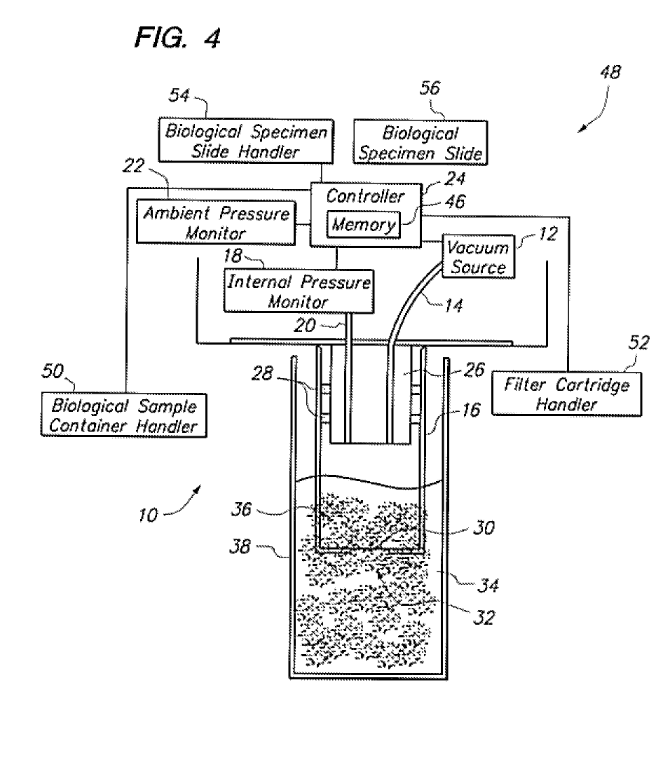

In operation, as shown in FIG. 4, the biological sample container handler 50, positions the biological sample container 38 and the filter cartridge handler 52 positions the filter cartridge 16 so that the open distal end 32 of the filter cartridge 16 is submerged in the liquid 34. This positioning also submerges the membrane 30 in the liquid 34.

The vacuum source 12 is then activated, generating a negative pressure in the filter cartridge 16. When the vacuum source 12 is activated, the controller 24 sends a signal to the internal pressure monitor 18, causing it to measure a first apparent pressure inside of the filter cartridge 16. The controller 24 also sends a signal to the door 44 in the ambient pressure monitor 22, causing the door 44 to close over the second opening 42, as shown in FIG. 2B, and activating the ambient pressure monitor 22, which measure a first ambient pressure.

The first apparent pressure and the first ambient pressure are communicated to the controller 24, which stores these first pressures along with a time value at which the pressures were measured in the memory 46. The internal pressure monitor 18 and the ambient pressure monitor 22 then measure a second set of apparent and ambient pressures, which are communicated to and stored in the controller 24 along with a second time value. The controller 24 calculates a change in ambient pressure by subtracting the first ambient pressure, or the baseline ambient pressure, from the second ambient pressure. The controller 24 also calculates a true pressure inside of the filter cartridge 16 by subtracting the change in ambient pressure from the second apparent pressure inside of the filter cartridge 16. The true pressure is stored in the memory 46. This measurement, storage, and calculation process is repeated until the true pressure falls below a predetermined number, for example 0.015 P.S.I.

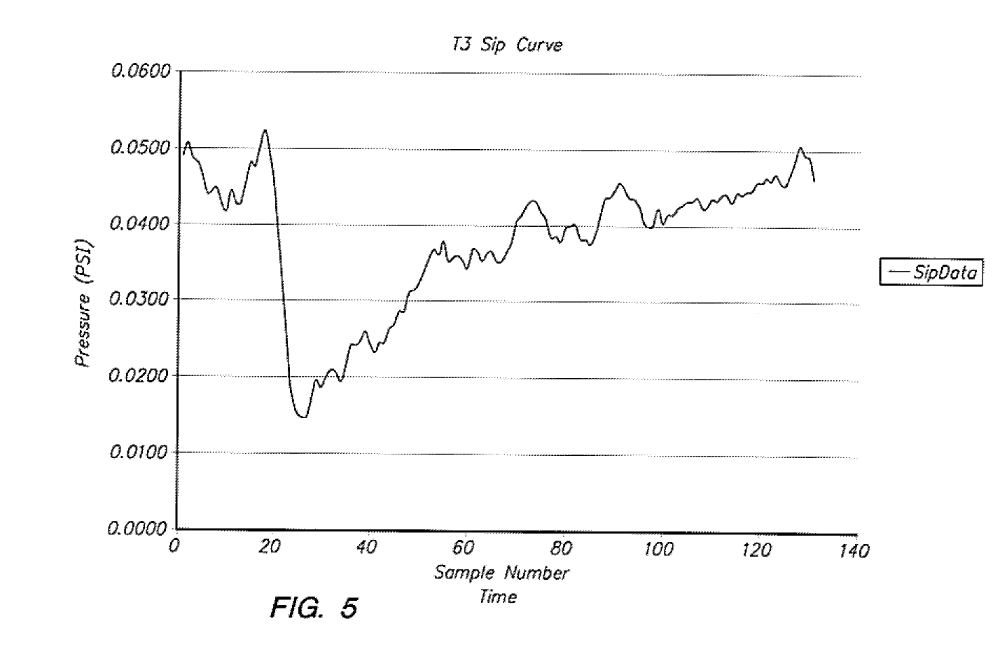

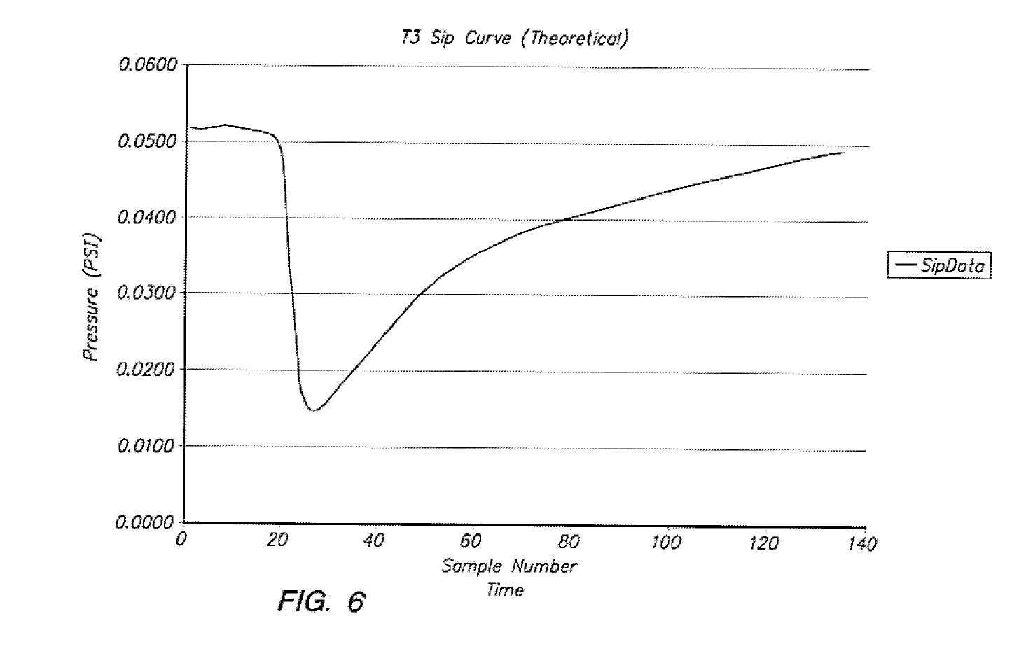

Then the controller 24 send a signal to deactivate the vacuum source 12. Then the pressure inside of the filter cartridge 16 equilibrates to ambient pressure as liquid 34 is drawn across the membrane 30 and into the filter cartridge 16. The measurement, storage, and calculation process continues during equilibration. The true pressures and time values generated are used to calculate a membrane occlusion percentage, using known methods. The values can also be plotted against each other, resulting in sip curves, as shown in FIGS. 5 and 6.

The biological specimen collection process is completed when the calculated membrane occlusion percentage reaches a predetermined value. By choosing this predetermined membrane occlusion percentage using known methods, an approximately one cell thick layer of cells is collected on the membrane 30. This layer of cells is then transferred to a biological specimen slide 56 using known methods.

FIG. 5 is a sip curve of the apparent pressure inside of the filter cartridge 16 over time. It is apparent that there is significant signal noise from changes in this curve, which complicates calculation of the membrane occlusion percentage. Accounting for changes in the ambient pressure minimizes the background noise from the sip curve and results in a curve that more resembles the theoretical sip curve shown in FIG. 6.

Although various embodiments of the invention have been shown and described herein, it should be understood that the above description and figures are for purposes of illustration only, and are not intended to be limiting of the invention, which is defined only by the appended claims and their equivalents.

What is claimed:

1. A filtration based biological specimen collection and transfer device, comprising:
a vacuum source;
a vacuum conduit configured to connect the vacuum source to a filter cartridge;
an internal pressure monitor configured to measure an apparent pressure inside of the filter cartridge;
an internal pressure monitor conduit configured to connect the internal pressure monitor to the filter cartridge;
an ambient pressure monitor configured to measure an ambient pressure; and
a controller operatively connected to the vacuum source, the internal pressure monitor, and the ambient pressure monitor.

2. The collection and transfer device of claim 1, wherein the controller is configured to calculate a true pressure inside of the filter cartridge based on a measured apparent pressure and a measured change in the ambient pressure.

3. The collection and transfer device of claim 1, the filter cartridge comprising:
a tubular body with a proximal end and a distal end; and
a membrane disposed on the distal end of the tubular body, wherein the proximal end is configured to form airtight seals with the vacuum conduit and the internal pressure monitor conduit.

4. The collection and transfer device of claim 3, wherein the controller is configured to calculate a true pressure inside of the filter cartridge based on a measured apparent pressure and a measured change in the ambient pressure, and to calculate a membrane occlusion percentage based on the calculated true pressure.

5. The collection and transfer device of claim 4, the controller comprising an associated memory, wherein the memory is configured to store true pressures calculated based on respective measured apparent pressures and respective measured changes in the ambient pressure, and the controller calculates the membrane occlusion percentage based on a change over time of the calculated true pressures.

6. The collection and transfer device of claim 1, the ambient pressure monitor comprising:
a first opening connecting the ambient pressure monitor to an ambient atmosphere;
a second opening connecting the ambient pressure monitor to the ambient atmosphere; and
a selectively closable door configured to close the second opening, wherein the ambient pressure monitor is configured to measure the ambient pressure at an instant when the door shuts.

7. The collection and transfer device of claim 6, wherein the controller is operatively connected to the door and configured to shut the door when the vacuum source is activated.

8. The collection and transfer device of claim 1, the controller comprising a memory configured to record a baseline ambient pressure measured by the ambient pressure monitor, wherein the controller is configured to calculate an ambient pressure change using the baseline ambient pressure and a respective measured ambient pressure.

9. A method of collecting a biological specimen, comprising:
providing a liquid biological sample in a biological sample container;
providing a filter cartridge forming a chamber and having a distal end with a membrane disposed thereon;
positioning the distal end of the filter cartridge and the membrane inside of the liquid biological sample in the biological sample container;
activating a vacuum source to lower the pressure of the chamber;
measuring an apparent pressure of the chamber;
measuring an ambient pressure; and
calculating a true pressure of the chamber using the respective measured apparent pressure and ambient pressure.

10. The method of claim 10, further comprising:
deactivating the vacuum source when the true pressure of the chamber falls below a predetermined pressure;
allowing the true pressure inside of the filter to return to approximately the ambient pressure; and
calculating a membrane occlusion percentage.

11. The method of claim 10, further comprising repeating the steps of claim 9 and claim 10 until the calculated membrane occlusion percentage is greater than a predetermined number.

12. The method of claim 11, further comprising:
measuring a baseline ambient pressure; and
calculating an ambient pressure change using the baseline ambient pressure and the respective ambient pressure.

13. The method of claim 11, further comprising:
storing true pressures calculated based on respective measured apparent pressures and respective measured changes in the ambient pressure; and
calculating over time the membrane occlusion percentage based on a change over time of the calculated true pressures.

14. The method of claim 11, further comprising collecting an approximately one cell thick layer of cells on the membrane.

15. The method of claim 14, further comprising transferring the layer of cells to a biological specimen slide.

16. A filtration based biological specimen collection and transfer system, comprising:
a biological sample container handler configured for use with a biological sample container;
a filter cartridge handler configured for use with a filter cartridge;
a biological specimen slide handler configured for use with a biological specimen slide;
a vacuum source;
a vacuum conduit configured to connect the vacuum source to a filter cartridge;
an internal pressure monitor configured to measure an apparent pressure inside of the filter cartridge;
an internal pressure monitor conduit configured to connect the internal pressure monitor to the filter cartridge;
an ambient pressure monitor configured to measure an ambient pressure; and
a controller operatively connected to the biological sample container handler; the filter cartridge handler, the biological specimen slide handler, the vacuum source, the internal pressure monitor, and the ambient pressure monitor.

17. The collection and transfer system of claim 16, wherein the controller is configured to calculate a true pressure inside of the filter cartridge based on a measured apparent pressure and a measured change in the ambient pressure.

18. The collection and transfer system of claim 16, the filter cartridge comprising:
a tubular body with a proximal end and a distal end; and
a membrane disposed on the distal end of the tubular body, wherein the proximal end is configured to form airtight seals with the vacuum conduit and the internal pressure monitor conduit.

19. The collection and transfer system of claim 18, wherein the controller is configured to calculate a true pressure inside of the filter cartridge based on a measured apparent pressure and a measured change in the ambient pressure, and to calculate a membrane occlusion percentage based on the calculated true pressure.

20. The collection and transfer device of claim 18, the controller comprising an associated memory, wherein the memory is configured to store true pressures calculated based on respective measured apparent pressures and respective measured changes in the ambient pressure, and the controller calculates the membrane occlusion percentage based on a change over time of the calculated true pressures.

21. The collection and transfer system of claim 16, the ambient pressure monitor comprising:
a first opening connecting the ambient pressure monitor to an ambient atmosphere;
a second opening connecting the ambient pressure monitor to the ambient atmosphere; and
a selectively closable door configured to close the second opening, wherein the ambient pressure monitor is configured to measure the ambient pressure at an instant when the door shuts.

22. The collection and transfer system of claim 20, wherein the controller is operatively connected to the door and configured to shut the door when the vacuum source is activated.

23. The collection and transfer system of claim 16, the controller comprising a memory configured to record a baseline ambient pressure measured by the ambient pressure monitor, wherein the controller is configured to calculate an ambient pressure change using the baseline ambient pressure and a respective measured ambient pressure.
